(19) United States
(12) Patent Application Publication
(10) Pub. No.: US 2008/0276356 A1
(43) Pub. Date: Nov. 13, 2008

(54) Ventilated safety goggle

(76) Inventor: Anthony Wang Lee, Yung-Kang City (TW)

Correspondence Address: ELIZABETH CHIEN-HALE, 40087 MISSION BLVD. BOX 367, FREMONT, CA, 94539, US

(21) Appl. No.: 11/801,623

(22) Filed: May 9, 2007

(51) Int. Cl. A61F 9/02

(52) U.S. Cl. 2/436

(57) ABSTRACT

A ventilated safety goggle has a lens and a frame. The frame is formed integrally, is mounted around an outer edge of the lens, extends rearward to form a protection cavity and has a circumferential wall encircling the protection cavity and at least one ventilation element. Each ventilation element has a cover and multiple air vents. The cover is formed on and protrudes outward from an outer surface of the circumferential wall of the frame and has a ventilation recess communicating with a rear of the cover. The air vents are formed through the circumferential wall of the frame and communicate the ventilation recess with the protection cavity. Air outside the frame can flow into/out of the protection cavity through the air vents to prevent a user's eyes from a muggy environment. Further, the frame can be easily manufactured by machines to improve a capacity of manufacturing the safety goggle.

### BACKGROUND OF THE INVENTION

1. Field of the Invention

The present invention relates to a safety goggle, and more particularly to a safety goggle that has improved ventilative effect and is convenient in assembly.

2. Description of Related Art

With reference to FIGS. 5 and 7, a conventional ventilated safety goggle in accordance with the prior art has a frame (80), a lens (91), multiple ventilation elements (70) and a binder (92). The frame (80) is resilient and has a front, a circumferential wall, a protection cavity, a mounting window and multiple mounting holes (81). The circumferential wall of the frame (80) has an inner surface and an outer surface. The protection cavity is formed inside the circumferential wall of the frame (80) to be mounted around a user's eyes. The lens (91) is mounted in the mounting window in the frame (80). The mounting holes (81) are formed through the circumferential wall of the frame (80).

With further reference to FIG. 6, the ventilation elements (70) are respectively mounted in the mounting holes (81) of the frame (80) and each ventilation element (70) has a tubular body (71), a bottom flange (72), a middle flange (73) and a top flange (75). The tubular body (71) has a closed top, an open bottom, a circumferential wall and multiple air vents (74). The open bottom of the tubular body (71) inserts into the protection cavity inside the frame (80). The air vents (74) are formed circularly through the circumferential wall of the tubular body (71) at intervals. Air outside the frame can flow into/out of the protection cavity inside the frame (80) through the air vents (74) to ventilate the protection cavity to protect the user's eyes from a muggy environment.

The bottom flange (72) of the ventilation element (70) is formed on and protrudes radially from the circumferential wall of the tubular body (71) near the open bottom of the tubular body (71), is larger than the mounting hole (81) of the frame (80) and abuts against the inner surface of the circumferential wall of the frame (80) to prevent the ventilation element (70) from detaching from the mounting hole (81). The middle flange (73) of the ventilation element (70) is formed on and protrudes radially from the circumferential wall of the tubular body (71) between the air vents (74) and the bottom flange (72), is larger than the mounting hole (81) of the frame (80) and abuts against the outer surface of the circumferential wall of the frame (80). Thus, the ventilation element (70) is retained in the mounting hole (81) by the middle flange (73) and the bottom flange (72).

The top flange (75) of the ventilation element (70) is formed on and protrudes radially from the circumferential wall of the tubular body (71) between the closed top and the air vents (74) to prevent hazardous matters, e.g. dusts, chemical splash, sparkles and the like from falling into the protection cavity through the air vents (74), so that the user's eyes are protected from the hazardous matters.

The binder (92) has two opposite ends attached to the circumferential wall of the frame (80) so that the user can wear the safety goggle on his/her head with the binder (92).

However, because the bottom flange (72) of the ventilation element (70) is larger than the mounting hole (81) of the frame (80), the bottom flange (72) has to be manually squeezed to pass through the mounting hole (81) to retain the ventilation element (70) in the mounting hole (81) with the middle flange (73), and this is time-consuming and inconvenient in assembly, and critically reduce a capacity of manufacture of the safety goggle.

### SUMMARY OF THE INVENTION

The primary objective of the present invention is to provide a ventilated safety goggle to mitigate or obviate the aforementioned problems.

The ventilated safety goggle in accordance with the present invention has a lens and a frame. The frame is resilient, is formed integrally, is mounted around an outer edge of the lens, extends rearward to form a protection cavity and has a circumferential wall encircling the protection cavity and at least one ventilation element. Each one of the at least one ventilation element has a cover and multiple air vents. The cover is formed on and protrudes outward from an outer surface of the circumferential wall of the frame and has a ventilation recess formed inside the cover and communicating with a rear of the cover. The air vents are formed through the circumferential wall of the frame and communicate the ventilation recess with the protection cavity.

Air outside the frame can flow into/out of the protection cavity through the air vents to prevent a user's eyes from a muggy environment. With the aforementioned configuration of the ventilation element, the frame is easily manufactured by machines without a procedure of assembling the ventilation element on the frame. Consequently, a capacity of manufacturing the safety goggle is significantly improved.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a ventilated safety goggle in accordance with the present invention with multiple air vents visible;

FIG. 2 is a cross sectional perspective view of the ventilated safety goggle in FIG. 1;

FIG. 3 is an enlarged cross sectional perspective view of the ventilated safety goggle in FIG. 1;

FIG. 4 is a perspective view of the ventilated safety goggle in FIG. 1 showing a front side of a frame; and

FIG. 5 is an exploded perspective view of a conventional ventilated safety goggle in accordance with the prior art with a ventilation element being detached;

FIG. 6 is an enlarged operational cross sectional rear view of the ventilated safety goggle in FIG. 5; and

FIG. 7 is a cross sectional rear view of the ventilated safety goggle in FIG. 5.

### DETAILED DESCRIPTION OF THE PREFERRED EMBODIMENT

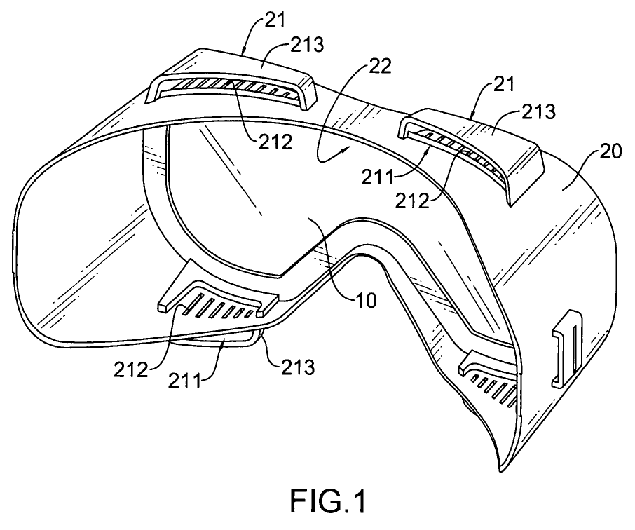

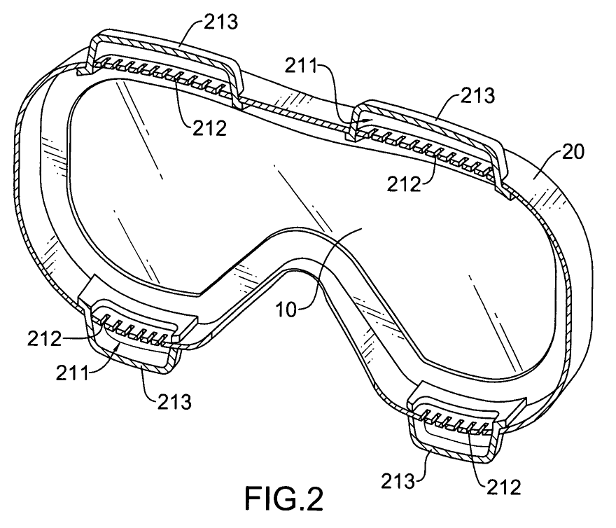

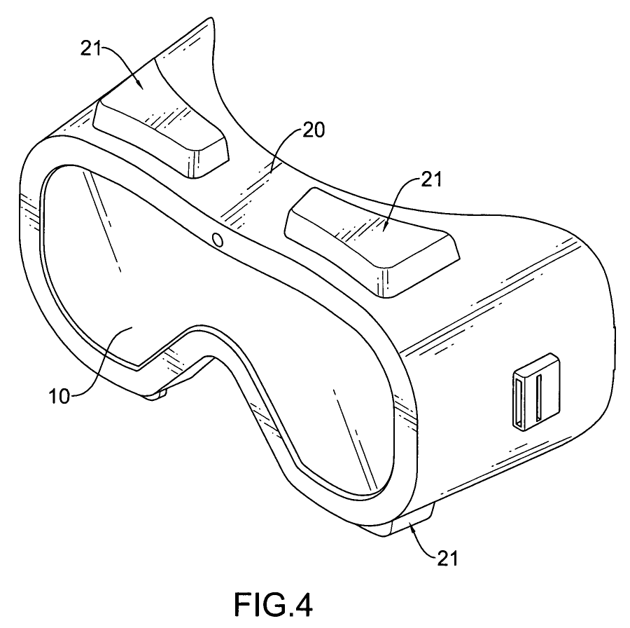

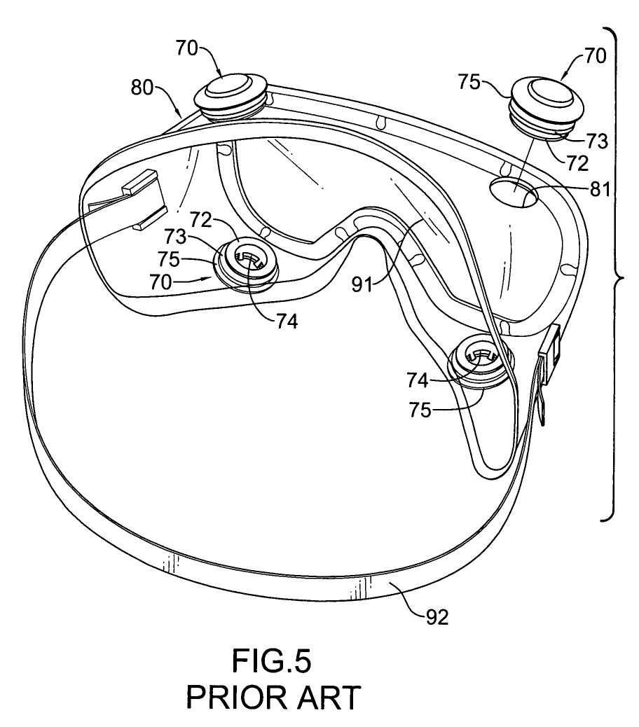

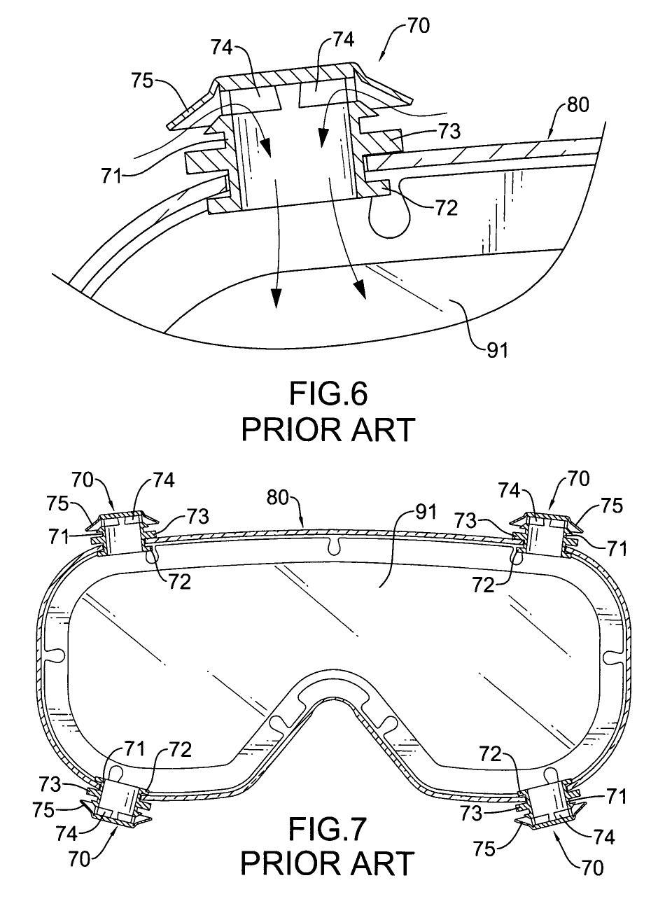

With reference to FIGS. 1, 2 and 4, a ventilated safety goggle in accordance with the present invention comprises a lens (10), a frame (20) and an optional binder. The lens (10) has an outer edge. The frame (20) is resilient, is formed integrally, is mounted around the outer edge of the lens (10) and extends rearward to form a protection cavity (22). The frame (20) has a circumferential wall and at least one ventilation element (21) and may be implemented in several ways. The circumferential wall of the frame (20) encircles the protection cavity (22) and has an outer surface. The at least one ventilation element (21) is formed on the circumferential wall of the frame (20) and each one of the at least one ventilation element (21) has a cover (213) and multiple air vents (212).

The cover (213) is formed on and protrudes outward from the outer surface of the circumferential wall of the frame (20) and has a rear and a ventilation recess (211). The ventilation recess (211) is formed inside the cover (213) and communicates with the rear of the cover (213). Because the ventilation recess (211) only communicates with the rear of the cover (213), hazardous matters, e.g. dusts, chemical splash, sparkles and the like are hard to fall into the ventilation recess (211).

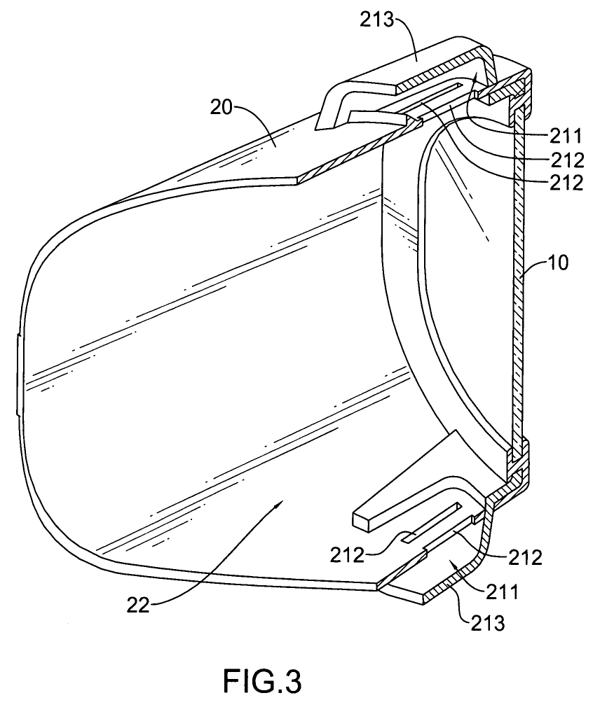

With further reference to FIG. 3, the air vents (212) are formed through the circumferential wall of the frame (20) and communicate the ventilation recess (211) inside the cover (213) with the protection cavity (22) inside the frame (20). So, air outside the frame (20) can flow into/out of the protection cavity (22) through the ventilation recess (211) and the air vents (212) to ventilate the protection cavity (22) to protect a user's eyes from muggy environment. Further, with the protection of the cover (213), hazardous matters are prevented from falling into the protection cavity (22) inside the frame (20) through the air vents (212) to protect the user's eyes from the hazardous matters. Additionally, the cover (213) hides the air vents (212) and the ventilation recess (211) to improve an appearance of the safety goggle.

In a preferred implementation of the frame (20), the frame (20) has multiple ventilation elements (21), such that air outside the frame (20) can flow into/out of the protection cavity (22) more efficiently.

The aforementioned configuration of the ventilation element (21) facilitates the frame (20) to be manufactured by machines, and this omits a procedure of assembling the ventilation element (21) on the frame (20) and significantly reduces the time of manufacturing the frame (20).

The lens (10) and the frame (20) may be integrally formed together by Co-Injection Molding manner. The Co-Injection Molding manner enables two compatible materials to be injection-molded into a mold cavity to form an integral product that has two parts with different material characteristics. Therefore, the resilient frame (20) can be directly and integrally mounted around the outer edge of the lens (20) by Co-Injection Molding manner to omit a procedure of assembling the frame (20) and the lens (10).

The binder has two opposite ends attached to the circumferential wall of the frame (80) so that the user can wear the safety goggle on his/her head with the binder (92).

Because the frame (20) can be directly and integrally formed around the outer edge of the lens (10), the safety goggle can be efficiently and massively produced to significantly increase a capacity of manufacturing the safety goggle.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A ventilated safety goggle comprising
a lens having an outer edge; and
a frame being resilient, being formed integrally, mounted around the outer edge of the lens, extending rearward to form a protection cavity and having
a circumferential wall encircling the protection cavity and having an outer surface; and
at least one ventilation element formed on the circumferential wall of the frame, and each one of the at least one ventilation element having
a cover formed on and protruding outward from the outer surface of the circumferential wall of the frame and having
a rear; and
a ventilation recess formed inside the cover and communicating with the rear of the cover; and
multiple air vents formed through the circumferential wall of the frame and communicating the ventilation recess inside the cover with the protection cavity inside the frame.

2. The ventilated safety goggle as claimed in claim 1, wherein the frame has multiple ventilation elements.

3. The ventilated safety goggle as claimed in claim 1, wherein the lens and the frame are integrally formed together.
